(19) United States
(12) Patent Application Publication
(10) Pub. No.: US 2021/0178161 A1
(43) Pub. Date: Jun. 17, 2021

(54) QUASI-ADIABATIC ELECTRICAL STIMULATOR

(71) Applicant: BIOTRONIK SE & Co. KG, Berlin (DE)

(72) Inventor: Marcelo BARU, Tualatin, OR (US)

(21) Appl. No.: 17/116,316

(22) Filed: Dec. 9, 2020

Related U.S. Application Data
(60) Provisional application No. 62/948,863, filed on Dec. 17, 2019.

(51) Int. Cl. A61N 1/36; A61N 1/04; A61N 1/05

(52) U.S. Cl. CPC A61N 1/36062; A61N 1/0456; A61N 1/36157; A61N 1/36053; A61N 1/36175; A61N 1/0551

(57) ABSTRACT

An electrical stimulation system includes a plurality of electrodes, a pulse generator (10) configured to generate a constant current pulse in a stimulation path between at least two electrodes of the plurality of electrodes during an active phase, where the pulse generator is configured to provide an output voltage overhead (V) for generating the constant current pulse. The output voltage (V) tracks a voltage ramp (V_{Track}) throughout the active phase, where the voltage ramp corresponds to a linear approximation of an accumulated voltage in an effective capacitance (C_{eff}) of the stimulation path throughout the active phase.

### TECHNICAL FIELD

The present invention relates to an electrical stimulation system having a pulse generator for providing electrical stimulation to a patient. The electrical stimulation system can be an implantable or non-implantable medical device for neurostimulation.

### BACKGROUND

The electrode-electrolyte (tissue) double-layer capacitive interface in electrical neurostimulation applications, e.g. spinal cord stimulation (SCS), may accumulate substantial voltage during an active phase (i.e. a phase where current is circulated through tissue, e.g. a stimulation phase) that needs to be accounted for to deliver a constant-current. Typically, the voltage overhead needed for current-based stimulation is programmed at a fixed value equal to the maximum value needed at the end of the active phase. Such maximum value is the summation of a minimum voltage needed for the current elements (that deliver stimulation) to operate maintaining a certain programmed constant current (compliance voltage), the ohmic drop in the electrolyte/tissue resistance caused by the stimulation current, the ohmic drop in connecting series analog switches, and the total accumulated voltage in the equivalent total capacitance in the stimulation path which may include traditional DC blocking capacitors for safety purposes besides the double-layer capacitances. Delivering stimulation with a fixed maximum voltage overhead throughout an active phase is inefficient.

Dynamic voltage overhead adaptation known in prior art, attempting to track the instantaneous electrode voltage to implement adiabatic stimulation, often suffer from excessive ripple in the output current and/or are not suitable for multi-current, multi-electrode simultaneous stimulation.

Electrical neurostimulation applications, in particular spinal cord stimulation (SCS), are demanding implantable pulse generator (IPG) architectures that can simultaneously source and sink currents from multiple electrodes, inject large charges, support high pulsing rates (thus active charge balancing), with reduced electrode areas (to improve selectivity) and without therapy interruption.

Unlike cardiac pacemakers, fractal coating of electrodes is not utilized in neurostimulation. SCS for example uses Pt/Ir electrodes which present a small electrode-electrolyte double-layer capacitance (in the μf range if linearized). In VNS, these capacitances are even smaller and in the order of a few hundred nF. These small capacitances, combined with the in series DC blocking capacitors (typically 10 μf), may result in substantial voltage accumulation during an active phase where current is circulated through tissue.

Typically, the voltage overhead required for current-based stimulation is programmed at the maximum level (which is really only required at the end of the active phase) which dissipates unnecessary power as the equivalent capacitance in the stimulation path charges.

Dynamic voltage overhead adaptation, to provide the minimum required overhead at any given time during the active phase to maintain the programmed currents constant (adiabatic stimulation), is desired in applications where the accumulated voltage in the equivalent capacitance is of comparable magnitude to the ohmic drop caused by the current in the electrolyte/tissue resistance. This permits minimizing power consumption thus extending the lifetime of the IPG.

Concepts for dynamic voltage overhead adaptation are presented for example in: [1] Kelly, S. K. and Wyatt, J. L., “A power-efficient neural tissue stimulator with energy recovery”, IEEE Transactions on Biomedical Circuits and Systems, 5(1):20-29, 2011; [2] Arfin, S. K. and Sarpeshkar, R., “An energy-efficient, adiabatic electrode stimulator with inductive energy recycling and feedback current regulation. IEEE Transactions on Biomedical Circuits and Systems, 6(1):14, 2012; [3] Williams, I. and Constandinou, T. G., “An energy-efficient, dynamic voltage scaling neural stimulator for a proprioceptive prosthesis”, IEEE Transactions on Biomedical Circuits and Systems, 7(2):10, 2013; and [4] Shirafkan, R. and Shoaei, O., “A power efficient, differential multichannel adiabatic electrode stimulator for deep brain stimulation”, Analog Integrated Circuits and Signal Processing, 95:481-497, 2018.

Stimulators known from prior art with dynamic power supplies typically use complex monitoring circuits. Kelly and Wyatt [1] propose a capacitor bank charged to different voltages that are switched sequentially to provide the required voltage overhead based on an RC-modelled load, where R is the ohmic drop in the stimulation path and C is the equivalent capacitance in such path. This switching can create ripple in the stimulation current which is undesired as the charge injected is uncontrolled.

In order to address the problem of discrete voltage steps of [1], Arfin and

Sarpeshkar [2] proposed an adiabatic stimulator which continuously controls the voltage across the electrode. In this prior art, the stimulator utilizes a dynamic power supply which provides a minimum voltage to make the current into the electrode constant during a stimulation phase by making use of current feedback. The output of the current sensor is a voltage proportional to the electrode current. Such control is indirect as the current sensor is not in series with the load. Hence, the control loop is based on the knowledge of the exact load impedance which unfortunately is complex given the stimulation chemical reactions. Further, the circuit is based on a step-down converter, limiting to low stimulation currents and uses a rather bulky capacitor (10 μf) as a midrail voltage reference at the output stage to make biphasic stimulation possible. The output current can present substantial ripple.

In Williams and Constandinou [3], a dynamic power supply technique, through monitoring the gate voltage of the transistor in a regulated cascade current sink, is proposed. The gate voltage of the outer transistor was monitored by comparators and a reference to provide an adjusting voltage. A reconfigurable switched capacitor DC-DC converter is used to provide four fixed output voltages.

Further, Shirafkan and Shoaei [4] utilize the same current sensing approach as presented in [2]. Although the differential power supply design disclosed removes the need for a midrail voltage, individual output filters capacitors are required for each channel.

Furthermore, US 2013/0338732 discloses an apparatus and method for providing efficient stimulation. Particularly, a switched mode power supply can be configured to generate a dynamic compliance voltage based on a stimulus waveform that can be non-rectangular. An output stimulation signal can be supplied to one or more outputs based on the compliance voltage.

Further, WO 2012/061608 A2 discloses an apparatus for providing efficient stimulation, wherein a variable compliance regulator can be connected to supply a compliance voltage to a power supply rail, which compliance voltage can vary dynamically based on a stimulus waveform.

Finally, WO 2004/052444 discloses a method for measuring impedance of a tissue, consisting of charging a capacitor to a potential, and discharging the capacitor for a discharge period through the tissue. The method further consists of measuring a voltage drop on the capacitor over the discharge period and determining the impedance of the tissue responsive to the potential, the voltage drop, and the discharge period.

### SUMMARY

Based on the above it is desirable to provide an improved pulse generator that allows (quasi) adiabatic stimulation in particular, and is particularly able to deliver the same stimulation current(s) as if a fixed voltage overhead were used (particularly no ripple, output current(s) can be assumed constant) throughout an active phase.

To this end an electrical stimulation system for providing electrical stimulation to a patient, particularly spinal cord stimulation (SCS) or vagus nerve stimulation (VNS) is disclosed according to claim 1, comprising: a plurality of electrodes, and a pulse generator configured to generate a constant current pulse in a stimulation path between at least two electrodes of said plurality of electrodes during an active phase, wherein the pulse generator is configured to provide an output voltage for generating the constant current pulse, which output voltage follows a voltage ramp throughout the active phase, wherein the voltage ramp corresponds to a linear approximation of an accumulated voltage in the effective capacitance of the stimulation path throughout the active phase (in the active phase current is circulated through tissue).

According to an embodiment of the proposed electrical stimulation system, the pulse generator is configured to generate a constant current pulse in a stimulation path between at least two electrodes of said plurality of electrodes and/or a metallic area of the pulse generator case during an active phase.

Advantageously, such a dynamic voltage overhead adaptation, particularly combined with charge self-balancing therapy, maximizes the service time of an implantable pulse generator (IPG). Exemplary systems and methods for an implantable medical device with self-balancing capabilities can be found for instance in US 2018/0272124 A1.

Particularly, the effective capacitance in the stimulation path is the sum of the capacitance of used DC blocking capacitors (if present) and the double-layer capacitances (due to electrode-tissue/electrolyte contact of the respective electrode).

According to an embodiment, the pulse generator comprises a step-up DC-to-DC voltage converter, configured to provide the output voltage overhead for generating the constant-current pulse(s), such that the output voltage ramps up from an initial output voltage until the end of the respective active phase.

Given the variability in compliance voltage of transistors typically employed to implement current elements (e.g. a transistor or a combination thereof) for stimulation, it is preferred to implement a step-up DC-to-DC voltage converter that continuously tracks a conservative representation of the voltage overhead ramp required for current stimulation throughout an active phase. A voltage ramp utilizing a 1/N (where N is preferably larger than 100) linear approximation of the capacitance in the stimulation path, and a 1/N of the maximum current amplitude programmed, is preferably self-generated in the step-up DC-to-DC voltage converter to create the required conservative voltage overhead. The system may be controlled using voltage comparators. Particularly, an output capacitor of the step-up DC-to-DC voltage converter is efficiently discharged in between active phases back to input voltage to permit consecutive voltage ramps during active phases.

According to an embodiment, the pulse generator is configured to measure at least a capacitance between the first and the second electrode for determining the initial output voltage.

According to an embodiment of the invention, the electrical stimulation system is an implantable medical device comprising the pulse generator. For instance, the implantable medical device is an implantable neurostimulation device for spinal cord stimulation (SCS), deep brain stimulation (DBS), vagus nerve stimulation (VNS), sacral nerve stimulation. According to an aspect, the implantable medical device is a cardiac rhythm management device, as e.g. a cardiac pacemaker, an implantable cardioverter-defibrillator (ICD), a cardiac resynchronization therapy (CRT) device, an implantable leadless pacemaker.

According to another aspect of the invention, the electrical stimulation system is an external medical device comprising the pulse generator according to the invention. The external medical device is for instance an external nerve stimulator, as a TENS (transcutaneous electrical nerve stimulation) device.

Further, according to an embodiment of the pulse generator according to the present invention, the pulse generator is configured to automatically determine the initial output voltage and a slope of the output voltage.

Furthermore, according to an embodiment of the electrical stimulation system, the pulse generator is configured to generate a plurality of constant current pulses (I, i=1, . . . ,N) in the stimulation path between electrodes of said plurality of electrodes during an active phase, wherein the pulse generator (particularly said step-up DC-to-DC voltage converter) is configured to provide said output voltage for generating the constant current pulses. As stated above, the output voltage follows said voltage ramp throughout the active phase, wherein the voltage ramp corresponds to a linear approximation of an accumulated voltage in a minimum effective capacitance of the stimulation path throughout the active phase.

Preferably, in an embodiment, the step-up DC-to-DC voltage converter comprises an output capacitor for providing the output voltage overhead required for current stimulation, wherein the output capacitor preferably comprises a capacitance below 300 nF, particularly below 200 nF, particularly below 100 nF. This enables the converter to quickly ramp the output voltage.

Further, in an embodiment, the step-up DC-to-DC voltage converter comprises an output voltage regulating feedback that is controlled by a first unit and a second unit of the pulse generator, wherein the first unit permits setting the initial output voltage, to be for example the expected maximum ohmic voltage drop in the stimulation path.

Particularly, according to an embodiment, the first unit can comprise a resistor digital-to-analog converter (DAC).

Further, in an embodiment, the pulse generator is configured to disconnect, before the respective active phase, the first unit from the step-up DC-to-DC voltage converter output and ground of the pulse generator, and to connect before the respective active phase the second unit, consisting of a current mirror connected to the mentioned output, to ground via a first resistor (R₁), a voltage follower (for example a PMOS or a bipolar transistor), and also to the ground via a second resistor (R₂) that provides the feedback (FB) signal.

According to an embodiment, the second unit forms a current mirror that imposes a first current through the first resistor (R₁) and a second current through the second resistor (R₂) to be equal, so that the output voltage V of the step-up DC-to-DC voltage converter follows the voltage ramp V_{Track} according to eq. (1):

V=V_{Track}+(R₁/R₂)×V_{FB}+V_{SG306}+V₃₀₄ (1)

where V_{FB} is the feedback's fixed voltage of the step-up DC-to-DC voltage converter (for example 1.2 V), V_{SG306} is a source-gate (or emitter-base) voltage of the voltage follower, and V₃₀₄ is a compliance voltage of the current mirror/second unit. Here, the compliance voltage is understood as the minimum output voltage required for the current mirror.

Particularly, the terms other than the voltage ramp V_{Track} in eq. (1) are preferably adjusted equal or higher than a minimum voltage required to guarantee the current amplitude I of the constant-current pulses is constant throughout the active phase. The first resistor R₁ is preferably set much smaller (ten times) than the second resistor R₂ to minimize the effect of the factor R₁/R₂ of V_{FB} in eq. (1).

According to an embodiment, the second unit comprises three transistors (for example bipolar or MOS transistors) connected to form a Wilson current mirror.

According to an embodiment, the first unit is configured to set the initial output voltage to a pre-defined voltage value (for example the required maximum ohmic voltage drop in the electrolyte/tissue resistance) and to periodically drop the initial voltage in discrete steps during therapy delivery until hitting said a compliance voltage of a current element to then permanently set the initial output voltage imposed by the first unit one step before hitting said minimal voltage. According to an alternative embodiment, the pulse generator can be configured to measure an excess offset voltage in the tracking at production and subtract it from a programmed initial output voltage.

In a preferred embodiment, the pulse generator comprises two H bridges and two diodes (preferably Schottky diodes) for self-generating the voltage ramp V_{Track} from the output voltage of the step-up DC-to-DC voltage converter.

Furthermore, according to an embodiment, the implantable pulse generator (IPG) is configured to provide spinal cord stimulation (SCS) and/or vagus nerve stimulation (VNS).

According to a further aspect of the present invention, a method for generating constant current stimulation pulses (particularly for SCS or VNS) is disclosed, the method comprising at least the step of: generating an output voltage overhead for generating a constant-current pulse in a simulation path between at least two electrodes and/or a metallic area of a pulse generator case during an active phase, wherein the output voltage overhead tracks a voltage ramp throughout the active phase, wherein the voltage ramp corresponds to a linear approximation of an accumulated voltage in an effective capacitance of the stimulation path throughout the active phase. Particularly, a plurality of constant-current pulses in a stimulation path between electrodes and/or a metallic area of a pulse generator case can be generated using said output voltage overhead.

Preferably, the method according to the present invention uses a pulse generator (IPG) according to the present invention.

### DESCRIPTION OF THE DRAWINGS

In the following, embodiments as well as further features and advantages of the present invention are described with reference to the Figures, wherein

FIG. 1 illustrates a constant-current pulse of amplitude I injected between two electrodes of a pulse generator;

FIG. 2 illustrates the use of a step-up DC-to-DC voltage converter according to an embodiment of a pulse generator according to the present invention, whose output voltage V (a variable V_{IStim} of FIG. 1) follows a voltage ramp to the end of the active phase to implement a quasi-adiabatic multi-electrode, multi-current Iᵢ (i=1 . . . N) stimulator to limit waste of energy during stimulation;

FIG. 3 shows a block diagram of an embodiment of a pulse generator according to the present invention comprising a step-up DC-to-DC voltage converter that tracks a voltage ramp V_{Track} throughout an active phase when constant-currents Iᵢ (i=1 . . . N) are delivered to the electrolyte/tissue as shown in FIG. 2;

FIG. 4 shows an embodiment of a second unit of the pulse generator of FIG. 3, which comprises bipolar (or MOS) transistors in a Wilson current mirror configuration;

FIG. 5 shows an embodiment of a detail of the pulse generator for self-generating the voltage ramp V_{Track} from the step-up DC-to-DC voltage converter output voltage utilizing two identical H-bridges;

FIG. 6 shows examples of the waveforms generated by the circuitry of FIG. 5; and

FIG. 7 shows an example of a stimulation constant-current pulse of 5.0 mA and 0.3 ms duration with output voltage overhead (V) adaptation.

### DETAILED DESCRIPTION

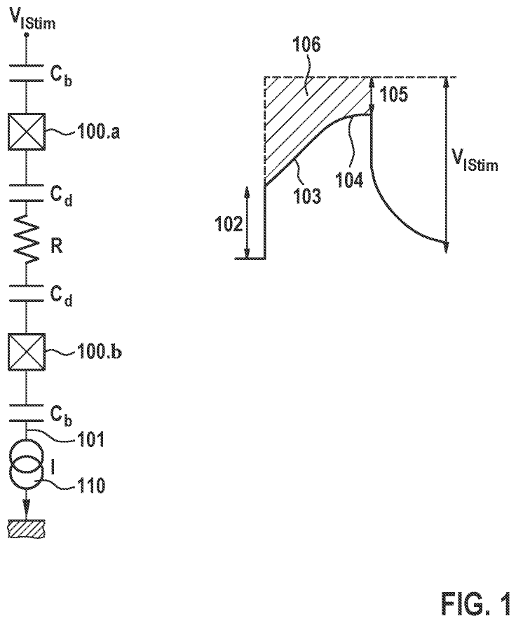

FIG. 1 illustrates a constant-current pulse of amplitude I injected between two electrodes 100.a and 100.b for stimulation at electrode 100.b. Elements C_{b} represent classical DC-blocking capacitors utilized in implantable pulse generators (IPGs) front-ends primarily for safety purposes. Element R represents the ohmic drop in the electrolyte(tissue) whereas elements C_{d} represent electrode-electrolyte(tissue) double-layer capacitances. As a current pulse of amplitude I is injected, the voltage difference between the total required voltage overhead V_{IStim} and node 101 has the profile shown on the right side of FIG. 1. Voltage drop 102 is the product of resistor R and current amplitude I. Then, the electrode (e.g. Pt/Ir) starts storing charge reversibly like a capacitor at the beginning of the active phase 103. As the electrode voltage increases, metal oxidation/reduction or other electrode reactions 104 may start to occur which causes a decrease in the voltage ramping 103 speed.

In fixed voltage-overhead DC-DC converter designs, voltage V_{IStim} is programmed equal to the sum of the voltage drops 102, 103, 104 and 105 where 105 is the compliance voltage of element 110 (e.g. transistor) that generates current amplitude I to be constant throughout the active phase. Hence, region 106 is a region of wasted energy in this design with fixed voltage overhead.

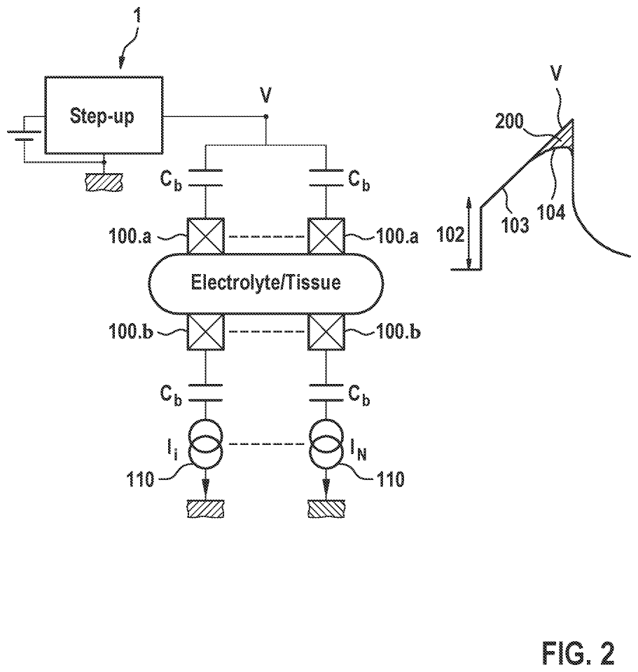

In the present Invention, a DC-to-DC voltage converter is proposed according to an embodiment, whose output voltage V (a variable V_{IStim}) starts at 102 and follows voltage ramp 103 to the end of the active phase as shown in FIG. 2 to implement a quasi-adiabatic multi-electrode 100, multi-current Iᵢ (i=1 . . . N) stimulator. With this design the amount of wasted energy is reduced to the region 200. The effective capacitance denominated C_{eff} that dictates voltage ramp 103 (basically the series of two C_{b} and two C_{d} elements considering FIG. 1) is determined for each electrical stimulation application. For an SCS application, for example, it can be observed that this C_{eff} capacitance is in the order of 1.5 μF. For a VNS application, on the other hand, this capacitance will be in the order of 680 nF. With stimulation amplitudes Iᵢ in the several mA and pulse widths of hundreds of μs, the accumulated voltage in C_{eff} can result in several volts and comparable to the ohmic voltage drop 102.

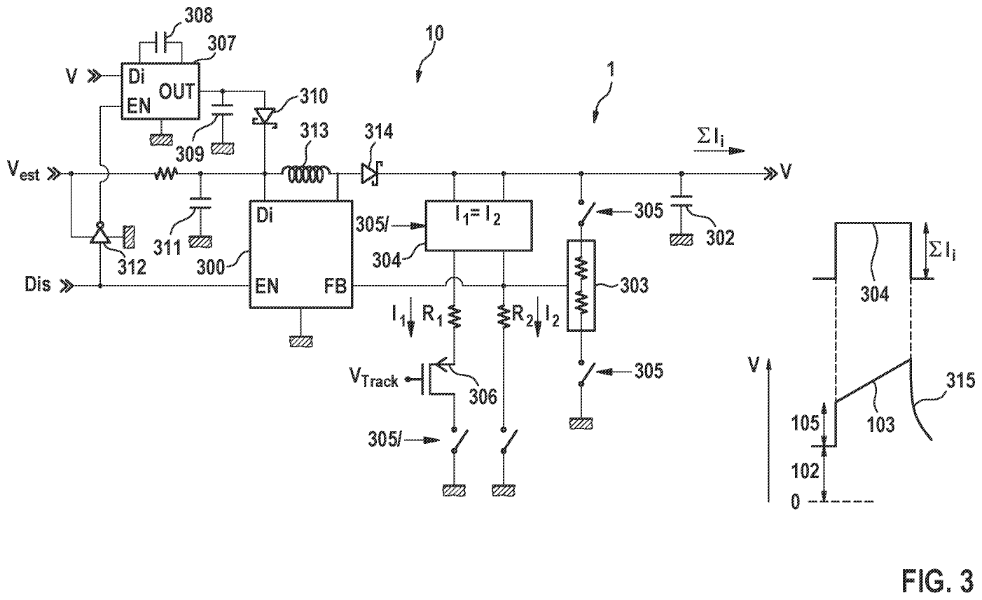

FIG. 3 shows a high-level block diagram of a preferred embodiment for the implementation of the step-up DC-DC voltage converter 300 that tracks a voltage ramp V_{Track} throughout active phase 301 when constant-currents Iᵢ (i=1 . . . N) are delivered to the electrolyte/tissue as shown in FIG. 2.

Unlike fixed-voltage output step-up designs, the step-up DC-to-DC voltage converter 300 has an output capacitor 302 in the order of only hundred(s) nF to quickly be able to ramp output voltage V. Step-up DC-to-DC voltage converter 300 preferably utilizes a pulse frequency modulation control scheme with adaptive constant on-time. The output-voltage regulating feedback FB of such step-up DC-to-DC voltage converter 300 is controlled by a first and a second unit 303, 304 depicted as blocks 303, 304 whose operation is mutually exclusive (by control signals 305 and 305/). The first unit/block 303 permits setting the expected maximum ohmic voltage drop 102 as initial output voltage V. Impedance measurements from any anode (i.e. an electrode 100.a connected via C_{b} to V in the example of FIG. 2) to any cathode (i.e. an electrode 100.b connected via C_{b} to a current element 110 of amplitude Iᵢ in the example of FIG. 2) is required to estimate such maximum ohmic voltage drop 102. An external programmer (for example the one that sets therapy) can calculate the maximum ohmic voltage drop 102 from these impedance measurements and pass the value back to the pulse generator 10 who will program the step-up 300.

The first unit/block 303 may be based on a resistor digital-to-analog converter (DAC). When it is time to deliver the first active phase 301, the first unit 303 is permanently disconnected, and the second unit 304, with voltage follower 306 (for example a PMOS or a bipolar transistor) and resistors R₁ and R₂, is connected. The second unit/block 304 is a current mirror that imposes current I₁ and I₂ (through resistors R₁ and R₂, respectively) to be equal. This implies the step-up DC-to-DC voltage converter 300 output voltage V will follow voltage V_{Track} according to eq. (1):

V=V_{Track}+(R₁/R₂)×V_{FB}+V_{SG306}+V₃₀₄ (1)

where V_{FB} is the feedback's fixed voltage of the step-up DC-to-DC voltage converter 300 (e.g. 1.2 V), V_{SG306} is the source-gate (or emitter-base) voltage of voltage follower 306, and V₃₀₄ is the compliance voltage of the current mirror 304. If V_{Track} provides the necessary voltage ramp 103, and the other terms in eq. (1) are adjusted equal (or larger) than voltage 105, then the circuit of FIG. 3 can provide quasi-adiabatic stimulation as illustrated in FIG. 2. R₁ is preferably set much smaller than R₂ (ten times) to minimize the effect of the term that multiplies V_{FB} in eq. (1).

Output capacitor 302 is preferably quickly discharged (for example for tens of μs) after an active phase 301 in preparation for the next active phase. Block 307, and associated components capacitors 308, 309 and Schottky diode 310, permit to efficiently discharge 315 the output capacitor 302 in preparation for the next active phase 301. Block 307 may preferably be a step-down charge pump back to input voltage V_{Bat} (e.g. battery voltage) to partially recover charge back to the input filter capacitor 311 of the step-up DC-to-DC voltage converter 300. The first unit 303 may be re-used, via extra analog switches not shown, to setup the output voltage of block 307.

The enabling of blocks 300 and 307 is mutually exclusive as illustrated by digital inverter 312. Inductor 313 and Schottky diode 314 are required for the operation of step-up DC-to-DC voltage converter 300. Schottky diode 314 may be replaced in a synchronous-rectifier step-up DC-to-DC voltage converter 300 design.

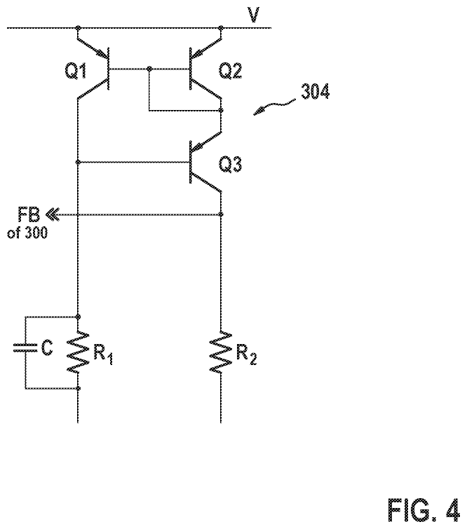

The second unit/block 304 may be implemented as shown in FIG. 4 utilizing bipolar (or MOS) transistors Q1, Q2, Q3 in a Wilson current mirror configuration. Capacitor C in parallel with R₁ can be used to minimize ripple on current I₁. In a preferred embodiment, R₁ is 47 kΩ, R₂ is 470 kΩ, and C is 470 pF. With this configuration, voltage V₃₀₄ in eq. (1) is equal to two emitter-base voltages, if bipolar transistors are employed, approximately 1.2 V. Considering V_{SG306} may be another 0.6 V, for example if a bipolar transistor is also used as voltage follower 306, the terms in eq. (1) other than V_{Track} may add up to 1.9 V or so of voltage offset in the tracking. Current elements 110 of amplitude may require a compliance voltage 105 of up to 1.0 V to deliver a constant current throughout an active phase. Hence, there is an excess of 0.9 V in output voltage V from the minimum required at the beginning of the active phase 301 (i.e. 102 plus 105) when tracking starts by second unit 304 taking over the control of the step-up DC-to-DC voltage converter 300 from first unit 303. In a preferred embodiment, as it will be described later, the first unit 303 may start by setting ohmic voltage drop 102 as the output voltage V and periodically drop the voltage (typically in discrete steps) during therapy delivery until hitting the compliance voltage 105 of a current element 110 to then permanently set the starting voltage imposed by the first unit 303 one step before hitting compliance. This voltage is denoted as 102adj. Alternatively, the excess offset voltage in the tracking is measured at production and subtracted from the programmed ohmic voltage drop 102 as described before.

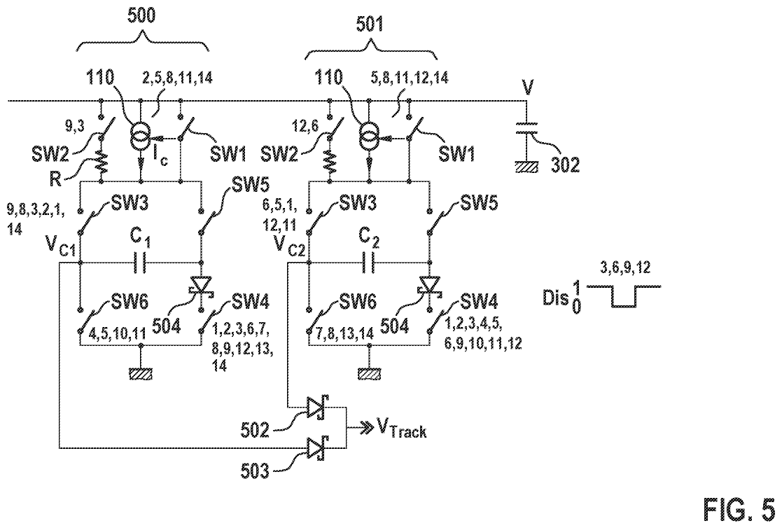

In a preferred embodiment, voltage V_{Track} is self-generated from the step-up DC-to-DC voltage converter 300 output voltage V utilizing two identical H bridges 500, 501, and Schottky diodes 502, 503 for example as shown in FIG. 5.

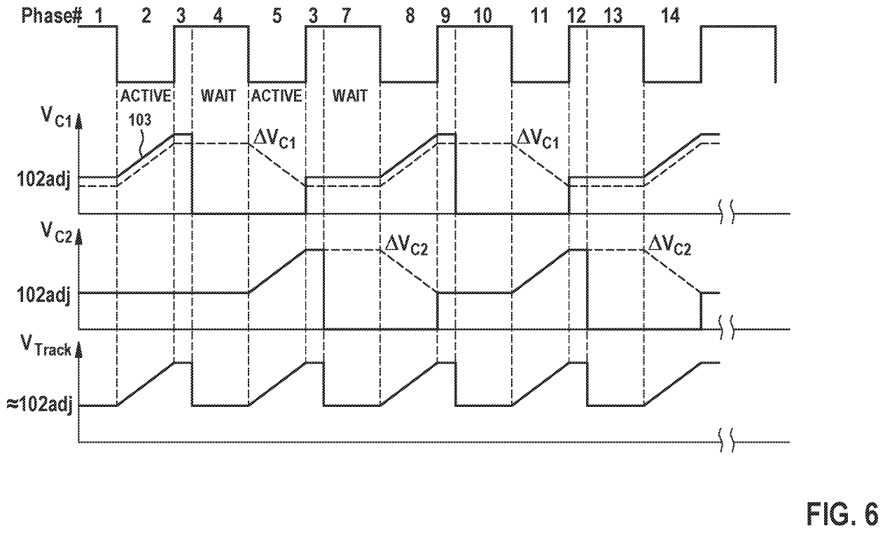

FIG. 6 depicts the desired waveforms for voltage tracking. Without losing generality, stimulation consists of phases of current delivery (ACTIVE) and pauses in between them (WAIT). These phases may have different durations, even same phases may have different durations. During a WAIT phase, passive charge balancing may be employed which does not consume power. As shown in FIG. 5, voltage V_{Track} is generated by the analog OR between voltages V_{C1} and V_{C2}, positive terminals of capacitors C₁ and C₂. These capacitors are selected equal to C_{effmin}/N, where N is preferably larger than 100 and C_{effmin} is the minimum expected capacitance in the stimulation path. For example, in SCS, C_{eff} may be 1.5 μf resulting in nominal C₁ and C₂ of 15 nF each.

Both capacitors C₁ and C₂ get initially charged to voltage 102adj using analog switches SW1, SW3, and SW4 when the first unit 303 provides feedback to step-up DC-to-DC voltage converter 300. During the first ACTIVE phase, switch SW1 is disconnected and capacitor C₁ charges via current I_{C} which is programmed as Iᵢₘₐₓ/N, where Iᵢₘₐₓ is the maximum programmed stimulation amplitude Iᵢ. Hence, the accumulated voltage in capacitor C₁ (ΔV_{C1}) increases with the required slope 103 and V_{Track} follows such ramp (as V_{C1} is higher than V_{C2}). Capacitor C₂ will undergo the same ramping in the next ACTIVE phase, whereas capacitor C₁ will discharge back to voltage 102adj during such phase. To discharge capacitor C₁ the same current I_{C} may be utilized and switches SW5 and SW6 closed instead of SW3 and SW4 (SW6 is closed during the previous WAIT phase and remains closed during the corresponding capacitor discharge). At the beginning of each WAIT phase, the step-up DC-to-DC voltage converter 300 is briefly disabled and its output capacitor 302 discharged via block 307 as described before. Also, during such time, current may be injected into capacitor C₁ (C₂) and resistor R via switches SW2, SW3 and SW4 to compensate for leakage to maintain the ramp up/down around voltage 102adj. Alternatively, different I_{C} currents can be used in the charging and discharging of C₁ (C₂) to compensate for leakage. For this alternative approach, just before the beginning of each ACTIVE phase, the output voltage V is sampled and held in a small capacitor. Once capacitor C₁ (C₂) undergoes a charge/discharge cycle, the resulting voltage V_{C1} (V_{C2}) is compared (via a comparator powered from output voltage V) against the sampled and held voltage and quickly corrected (either using SW2 and resistor R, or a reduced current I_{C} and analog switches SW3 and SW4) during the beginning of the corresponding WAIT phase. Schottky diodes 504 prevent reverse conduction through switches SW4 when V_{C1} (V_{C2}) is brought to ground during the corresponding WAIT plus ACTIVE phases.

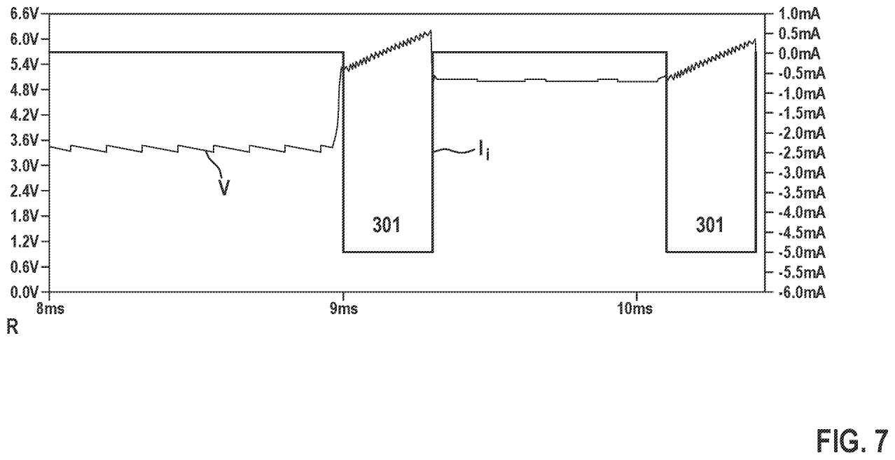

FIG. 7 shows the example of simulation pulses 301 of 5.0 mA, 0.3 ms. As it can be seen, the step-up DC-to-DC voltage converter 300 output voltage V ramps up with a certain ripple but the stimulation amplitude Iᵢ can be assumed constant as desired.

In a preferred method, the step-up DC-to-DC voltage converter 300 automatically finds the optimum initial voltage 102adj (to be imposed by first unit 303) and the minimum required slope 103 to account for component variabilities in the design. To find the optimum initial voltage 102adj, the maximum ohmic voltage 102 is programmed first and current I_{C} programmed with a smaller N than required which generates a slope faster than the required 103 considering capacitor's C₁ (C₂) tolerance and the accuracy of current I_{C}. For example, if C₁ (C₂) has a tolerance of ±10% and the accuracy of I_{C} is ±5%, N can be initially programmed 20% or smaller than required for finding the initial output voltage 102adj. After 60 s of delivering stimulation (steady-state reached), the first unit 303 may be re-connected (during a WAIT phase) to generate an output voltage V one step below (e.g. 0.5 V) the original programmed ohmic voltage 102 and monitor if any of the compliance voltages 105 of a current element 110 is reached. If not, consecutive voltage drops may be generated until a compliance voltage 105 is reached and then permanently setting the initial output voltage 102adj imposed by first unit 303 one step before hitting compliance to set the optimum voltage 102adj.

Once the optimum initial output voltage 102adj is found, N is also increased in steps (to reduce the slope 103) until reaching a compliance voltage 105 and then permanently programming N one step before hitting compliance to set the optimum slope 103.

Step-up DC-to-DC voltage converter 300 may be designed with dual output where only one is active at any time. The second output may be with a constant voltage (set via first unit 303) and a larger output capacitor 302.

The disclosed quasi-adiabatic electrical stimulator is suitable for simultaneous multi-electrode, multi-current stimulation. It reduces power consumption. Unlike prior art, it maintains constant-current (i.e. no ripple) throughout active phases. Further, it utilizes smaller capacitors which may allow integrated passive device technology to be employed to implement these capacitors thus reducing size.

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

What is claimed is:

1. An electrical stimulation system comprising:
(a) a plurality of electrodes; and
(b) a pulse generator configured to generate a constant current pulse in a stimulation path between at least two electrodes of said plurality of electrodes during an active phase;
wherein the pulse generator is configured to provide an output voltage (V) for generating the constant current pulse, which output voltage (V) tracks a voltage ramp (V_{Track}) throughout the active phase, wherein the voltage ramp corresponds to a linear approximation of an accumulated voltage in an effective capacitance (C_{eff}) of the stimulation path throughout the active phase.

2. The electrical stimulation system of claim 1, wherein the pulse generator comprises a step-up DC-to-DC voltage converter configured to provide the output voltage (V), such that the output voltage (V) ramps up from an initial output voltage until the end of the active phase.

3. The electrical stimulation system of claim 2, wherein the pulse generator is configured to measure the ohmic component between at least two electrodes for determining the initial output voltage.

4. The electrical stimulation system of claim 2, wherein the pulse generator is configured to automatically determine the initial output voltage and a slope of the output voltage (V).

5. The electrical stimulation system of claim 2, wherein the step-up DC-to-DC voltage converter comprises an output capacitor for providing the output voltage (V) required for current stimulation.

6. The electrical stimulation system of claim 2, wherein the step-up DC-to-DC voltage converter is configured to employ an output voltage regulating feedback (FB) that is controlled by a first unit and a second unit of the pulse generator, wherein the first unit permits setting the initial output voltage (V).

7. The electrical stimulation system of claim 6, wherein the first unit comprises a resistor digital-to-analog converter (DAC).

8. The electrical stimulation system of claim 6, wherein the pulse generator is configured to:
(i) before the active phase, disconnect the first unit from the output voltage (V) and ground of the pulse generator,
(ii) before the active phase, connect the second unit, that is connected to the output voltage (V), to the ground via a first resistor (R₁) and a voltage follower, and
(iii) connect to the ground via a second resistor (R₂) that provides the feedback (FB) signal.

9. The electrical stimulation system of claim 6, wherein the second unit forms a current mirror that imposes a first current (I₁) through the first resistor (R₁) and a second current (I₂) through the second resistor (R₂) to be equal.

10. The electrical stimulation system of claim 6, wherein the second unit comprises three transistors (Q1, Q2, Q3) connected to form a Wilson current mirror.

11. The electrical stimulation system of claim 2, wherein the pulse generator comprises two H bridges and two diodes for self-generating the voltage ramp (V_{Track}) from the output voltage (V) of the step-up DC-to-DC voltage converter).

12. The electrical stimulation system of claim 1, wherein the pulse generator is configured to generate a plurality of constant current pulses (Iᵢ, i=1, . . . ,N) in the stimulation path between electrodes of said plurality of electrodes during an active phase, wherein the pulse generator is configured to provide said output voltage (V) for generating the constant current pulses, which output voltage (V) tracks said voltage ramp (V_{Track}) throughout the active phase, wherein the voltage ramp (V_{Track}) corresponds to a linear approximation of an accumulated voltage in an effective capacitance (C_{eff} ) of the stimulation path throughout the active phase.

13. The electrical stimulation system of claim 1, wherein the electrical stimulation system is an implantable medical device or a part thereof, wherein the implantable medical device is configured to provide spinal cord stimulation (SCS) and/or vagus nerve stimulation (VNS).

14. A method for generating constant-current stimulation pulses, comprising at least the step of:
generating a variable output voltage overhead (V) for generating a constant-current pulse in a simulation path between at least two electrodes during an active phase, wherein the output voltage overhead (V) tracks a voltage ramp (V_{Track}) throughout the active phase, wherein the voltage ramp (V_{Track}) corresponds to a linear approximation of an accumulated voltage in an effective capacitance (C_{eff}) of the stimulation path throughout the active phase.
